(19) United States
(12) Patent Application Publication
(10) Pub. No.: US 2017/0071869 A1
(43) Pub. Date: Mar. 16, 2017

(54) TRANSMUCOSAL DELIVERY OF LAQUINIMOD BY ORAL PATCHES

(71) Applicants: Ralph Stefan, Ebenweiler (DE); Hans Juergen Mika, Bonn (DE); Claudia Lauer, Munich (DE); Dirk Schenk, Munich (DE); Sabine Prohl, Munich (DE)

(72) Inventors: Ralph Stefan, Ebenweiler (DE); Hans Juergen Mika, Bonn (DE); Claudia Lauer, Munich (DE); Dirk Schenk, Munich (DE); Sabine Prohl, Munich (DE)

(73) Assignee: Teva Pharmaceutical Industries Ltd., Petach Tikva (IL)

(21) Appl. No.: 15/123,208

(22) PCT Filed: Mar. 13, 2015
(86) PCT No.: PCT/US2015/020432

§ 371 Date: Sep. 1, 2016

Related U.S. Application Data
(60) Provisional application No. 61/953,552, filed on Mar. 14, 2014.

(51) Int. Cl. A61K 9/70; A61K 31/4704; A61K 9/00

(52) U.S. Cl. CPC A61K 9/70; A61K 9/006; A61K 31/4704

(57) ABSTRACT

The subject invention provides an oral patch comprising: a) a liner; and b) a film composition thereon, the film composition comprising (i) laquinimod in an amount of about 0.1%-20% by weight of the film composition, and (ii) one or more film forming agents in a total amount of about 40%-90% by weight of the film composition. The subject invention also provides a method for delivering laquinimod across the oral mucosa of a subject, or for treating a human subject afflicted with a form of multiple sclerosis, comprising periodically administering to the human subject an oral patch as described herein. The subject invention also provides an oral patch as described herein for use in treating a human subject afflicted with a form of multiple sclerosis.

This application claims the priority of U.S. Provisional Application No. 61/953,552, filed Mar. 14, 2014, the contents of which are hereby incorporated by reference.

Throughout this application, various publications are referred to by first author and year of publication. Full citations for these publications are presented in a References section immediately before the claims. Disclosures of the documents and publications referred to herein are hereby incorporated in their entireties by reference into this application.

### BACKGROUND OF THE INVENTION

### Multiple Sclerosis

Multiple Sclerosis (MS) is a neurological disease affecting more than 1 million people worldwide. It is the most common cause of neurological disability in young and middle-aged adults and has a major physical, psychological, social and financial impact on subjects and their families, friends and bodies responsible for health care (EMEA Guideline, 2006).

A clinically isolated syndrome (CIS) is a single monosymptomatic attack suggestive of MS, such as optic neuritis, brain stem symptoms, and partial myelitis. Patients with CIS that experience a second clinical attack are generally considered to have clinically definite multiple sclerosis (CDMS). Various MS disease stages and/or types are described in Multiple Sclerosis Therapeutics (Duntiz, 1999). Among them, relapsing-remitting multiple sclerosis (RRMS) is the most common form at the time of initial diagnosis. Many subjects with RRMS have an initial relapsing-remitting course for 5-15 years, which then advances into the secondary progressive MS (SPMS) disease course. There are currently a number of disease-modifying medications approved for use in relapsing MS (RMS), which includes RRMS and SPMS (The Disease Modifying Drug Brochure, 2006). These include interferon beta 1-a (Avonex® and Rebif®), interferon beta 1-b (Betaseron®), glatiramer acetate (Copaxone®), mitoxantrone (Novantrone®), natalizumab (Tysabri®) and Fingolimod (Gilenya®). Immunosuppressants or cytotoxic agents are used in some subjects after failure of conventional therapies. However, the relationship between changes of the immune response induced by these agents and the clinical efficacy in MS is far from settled (EMEA Guideline, 2006).

Other therapeutic approaches include symptomatic treatment which refers to all therapies applied to improve the symptoms caused by the disease (EMEA Guideline, 2006) and treatment of acute relapses with corticosteroids. While steroids do not affect the course of MS over time, they can reduce the duration and severity of attacks in some subjects.

### Laquinimod

Laquinimod is a novel synthetic compound with high oral bioavailability which has been suggested as an oral formulation for the treatment of Multiple Sclerosis (MS) (Polman, 2005; Sandberg-Wollheim, 2005). Laquinimod and its sodium salt form are described in, for example, U.S. Pat. No. 6,077,851.

The mechanism of action of laquinimod is not fully understood. Animal studies show it causes a Th1 (T helper 1 cell, produces pro-inflammatory cytokines) to Th2 (T helper 2 cell, produces anti-inflammatory cytokines) shift with an anti-inflammatory profile (Yang, 2004; Brück, 2011). Another study demonstrated (mainly via the NFkB pathway) that laquinimod induced suppression of genes related to antigen presentation and corresponding inflammatory pathways (Gurevich, 2010).

Laquinimod showed a favorable safety and tolerability profile in two phase III trials (Results of Phase III BRAVO Trial Reinforce Unique Profile of Laquinimod for Multiple Sclerosis Treatment; Teva Pharma, Active Biotech Post Positive Laquinimod Phase 3 ALLEGRO Results).

### Advantages of Mucoadhesive Buccal Drug Delivery System

Buccal administration avoids hepatic metabolism and gastrointestinal degradation which can hinder effectiveness of orally administered drugs and provides an attractive alternative to oral administration. Drugs administered via oral mucosa offers additional advantages:
1. Ease of administration, since the drug can be taken without water, leading to higher patient compliance.
2. Ease of therapy termination.
3. Fast release.
4. Permits localization of drug to the oral cavity for a prolonged period of time.
5. Can be administered to unconscious patients.
6. Offers an excellent route, for the systemic delivery of drugs with high first pass metabolism, thereby offering a greater bioavailability.
7. A significant reduction in dose can be achieved thereby reducing dose related side effects.
8. Drugs which are unstable in the acidic environment (destroyed by enzymatic or alkaline environment of intestine) can be administered by this route.
9. Drugs which show poor bioavailability via the oral route can be administered conveniently.
10. Offers a passive system of drug absorption and does not require any activation.
11. The presence of saliva ensures relatively large amount of water for drug dissolution unlike in case of rectal and transdermal routes.
12. Systemic absorption is rapid.
13. Provides an alternative for the administration of various hormones, narcotic analgesic, steroids, enzymes, cardiovascular agents etc.
14. The buccal mucosa is highly perfused with blood vessels and offers a greater permeability than the skin.

### SUMMARY OF THE INVENTION

The subject invention provides an oral patch comprising: a) a liner; and b) a film composition thereon, the film composition comprising (i) laquinimod in an amount of about 0.1%-20% by weight of the film composition, and (ii) one or more film forming agents in a total amount of about 40%-90% by weight of the film composition.

The subject invention also provides an oral patch comprising: a) a PET liner, and b) a film composition thereon, the film composition comprising (i) laquinimod in an amount of about 1% by weight of the film composition, (ii) hydroxypropylcellulose present in the film composition in an amount of about 7% by weight of the film composition, (iii) polyethylene glycol present in the film composition in an amount of about 10% by weight of the film composition, (iv) microcrystalline cellulose present in the film composition in an amount of about 44% by weight of the film composition, (v) sorbitol present in the film composition in an amount of about 36% by weight of the film composition, and (vi) acesulfam present in the film composition in an amount of about 1.4% by weight of the film composition.

The subject invention also provides an oral patch comprising: a) a PET liner; and b) a film composition thereon, the film composition comprising (i) laquinimod in an amount of about 1% by weight of the film composition, (ii) copovidone present in the film composition in an amount of about 43% by weight of the film composition, (iii) polyethylene glycol present in the film composition in an amount of about 6% by weight of the film composition, (iv) starch present in the film composition in an amount of about 20% by weight of the film composition, v) hydroxyethylcellulose present in the film composition in an amount of about 3% by weight of the film composition, vi) sorbitol present in the film composition in an amount of about 26% by weight of the film composition, and vii) acesulfam present in the film composition in an amount of about 1.4 percent by weight of the film composition.

The subject invention also provides an oral patch comprising: PET liner; and b) a film composition thereon, the film composition comprising (i) laquinimod in an amount of about 1% by weight of the film composition, (ii) polyvinyl alcohol present in the film composition in an amount of about 43% by weight of the film composition, (iii) polyethylene glycol present in the film composition in an amount of about 9% by weight of the film composition, (iv) starch present in the film composition in an amount of about 20% by weight of the film composition, (v) carbomer present in the film composition in an amount of about 0.7% by weight of the film composition, (vi) sorbitol present in the film composition in an amount of about 26% by weight of the film composition, and (vii) acesulfam present in the film composition in an amount of about 1.4 percent by weight of the film composition.

The subject invention also provides a method for delivering laquinimod across the oral mucosa of a subject comprising administering to the oral mucosa of the subject an oral patch as described herein.

The subject invention also provides a method for treating a human subject afflicted with a form of multiple sclerosis, comprising periodically administering to the human subject an oral patch as described herein.

The subject invention also provides an oral patch as described herein for use in treating a human subject afflicted with a form of multiple sclerosis.

### BRIEF DESCRIPTION OF THE DRAWINGS

FIG. 1: Graph showing dissolution profile of laquinimod oral film according to Examples 1-3 vs. 0.6 mg laquinimod capsule.

### DETAILED DESCRIPTION OF THE INVENTION

### Embodiments of the Invention

The subject invention provides an oral patch comprising: a) a liner; and b) a film composition thereon, the film composition comprising (i) laquinimod in an amount of about 0.1%-20% by weight of the film composition, and (ii) one or more film forming agents in a total amount of about 40%-90% by weight of the film composition.

In one embodiment, laquinimod is present in the film composition in an amount of about 0.2%-10% by weight of the film composition. In another embodiment, laquinimod is present in the film composition in an amount of about 0.6%-8% percent by weight of the film composition. In another embodiment, laquinimod is present in the film composition in an amount of about 0.7%-1.5% by weight of the film composition.

In an embodiment, the one or more film forming agents are present in the film composition in a total amount of about 60%-80% by weight of the film composition. In another embodiment, the one or more film forming agents are selected from the group consisting of Carbomer (sodium salt), polyethylene glycol, polyvinyl alcohol, microcrystalline cellulose, starch, hydroxypropyl methylcellulose, amylopectin, ethylcellulose, gelatine, hydroxypropylcellulose, hydroxyethylcellulose, methylcellulose, carboxymethylcellulose, gummi arabicum, xanthan gum, carrageen, povidone, copovidone.

In one embodiment, one or more film forming agents comprises carbomer (sodium salt), present in the film composition in an amount of about 0.1%-1% by weight of the film composition. In another embodiment, the one or more film forming agents comprises polyvinyl alcohol, present in the film composition in an amount of about 30%-50% by weight of the film composition. In another embodiment, the one or more film forming agents comprises microcrystalline cellulose, present in the film composition in an amount of about 30%-50% by weight of the film composition. In another embodiment, the one or more film forming agents comprises copovidone, present in the film composition in an amount of about 30%-50% by weight of the film composition. In another embodiment, the one or more film forming agents comprises starch, present in the film composition in an amount of about 10%-30% by weight of the film composition. In another embodiment, the one or more film forming agents comprises polyethylene glycol, present in the film composition in an amount of about 5%-15% by weight of the film composition. In another embodiment, the one or more film forming agents comprises hydroxyethyl cellulose, present in the film composition in an amount of about 1%-10% by weight of the film composition. In another embodiment, the one or more film forming agents comprises hydroxypropyl cellulose, present in the film composition in an amount of about 1%-10% by weight of the film composition. In another embodiment, the film composition further comprises one or more fillers, present in the film composition in a total amount of about 10%-50% by weight of the film composition. In yet another embodiment, the one or more fillers are present in the film composition in an amount of about 20%-40% by weight of the film composition.

In one embodiment, the one or more fillers are selected from the group consisting of sorbitol, lactose, saccharose, sucrose, dextrose, isomalt calcium phosphate, calcium carbonate, calcium silicate, magnesium carbonate, magnesium oxide, glucopyranosyl mannitol and calcium sulfate. In another embodiment the one or more fillers comprises sorbitol, present in the film composition in an amount of about 20%-40% by weight of the film composition.

In one embodiment, the film composition further comprises one or more flavorants, present in the film composition in a total amount up to about 10% by weight of the film composition. In another embodiment, the flavorants are selected from the group consisting of acesulfam, saccharin-sodium, aspartame, stevia, spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, cassia oil, vanilla, ethyl vanillin, citrus oils, lemon oil, orange oil, tangerine oil, lime oil, grapefruit oil, apple flavor, pear flavor, peach flavor, orange flavor, grape flavor, strawberry flavor, raspberry flavor, cherry flavor, plum flavor, pineapple flavor, apricot flavor, cinnamyl acetate, cinnamaldehyde, citral diethylacetal, dihydrocarvyl acetate, eugenyl formate and p-methylamisol. In another embodiment, the one or more flavorants comprises acesulfam, present in the film composition in an amount of about 1%-3% by weight of the film composition.

In one embodiment, the film composition further comprises one or more permeation enhancers, present in the film composition in a total amount up to about 10% by weight of the film composition. In another embodiment, the one or more permeation enhancers are selected from the group consisting of DMSO, n-Dodecyl nitrogen heterocyclic heptane-2-ketone, propylene glycol, oleic acid, isopropylmyristat and d,l-alpha-toccopherol.

In one embodiment, the film composition further comprises a pigment, present in the film composition in an amount up to about 5% by weight of the film composition. In another embodiment, the pigment is selected from the group consisting of titanium dioxide, talc and ferric oxide.

In one embodiment, the film composition further comprises one or more humectants, present in the film composition in an amount up to about 5% by weight of the film composition.

In one embodiment, the patch has an area about 5-15 cm². In another embodiment, the patch has an area about 10 cm².

In one embodiment, the patch comprises about 0.25-7.5 mg laquinimod. In another embodiment, the patch comprises about 0.75 mg laquinimod.

In one embodiment, the patch comprises about 0.5-5 mg laquinimod per 10 cm² of patch area. In another embodiment, the patch comprises about 0.75 mg laquinimod per 10 cm² of patch area.

In one embodiment, the liner is a polyethylene terephthalate (PET) liner.

In an embodiment, the amount of laquinimod present in the pharmaceutical composition is a least laquinimod's saturation amount. In another embodiment, the amount of laquinimod present in the pharmaceutical composition is higher than laquinimod's saturation amount.

The subject invention also provides an oral patch comprising: a PET liner, and b) a film composition thereon, the film composition comprising (i) laquinimod in an amount of about 1% by weight of the film composition, (ii) hydroxypropylcellulose present in the film composition in an amount of about 7% by weight of the film composition, (iii) polyethylene glycol present in the film composition in an amount of about 10% by weight of the film composition, (iv) microcrystalline cellulose present in the film composition in an amount of about 44% by, weight of the film composition, (v) sorbitol present in the film composition in an amount of about 36% by weight of the film composition, and (vi) acesulfam present in the film composition in an amount of about 1.4% by weight of the film composition.

The subject invention also provides an oral patch comprising: a) a PET liner; and b) a film composition thereon, the film composition comprising (i) laquinimod in an amount of about 1% by weight of the film composition, (ii) copovidone present in the film composition in an amount of about 43% by weight of the film composition, (iii) polyethylene glycol present in the film composition in an amount of about 6% by weight of the film composition, (iv) starch present in the film composition in an amount of about 20% by weight of the film composition, v) hydroxyethylcellulose present in the film composition in an amount of about 3% by weight of the film composition, vi) sorbitol present in the film composition in an amount of about 26% by weight of the film composition, and vii) acesulfam present in the film composition in an amount of about 1.4 percent by weight of the film composition.

The subject invention also provides an oral patch comprising: a) a PET liner; and b) a film composition thereon, the film composition comprising (i) laquinimod in an amount of about 1% by weight of the film composition, (ii) polyvinyl alcohol present in the film composition in an amount of about 43% by weight of the film composition, (iii) polyethylene glycol present in the film composition in an amount of about 9% by weight of the film composition, (iv) starch present in the film composition in an amount of about 20% by weight of the film composition, (v) carbomer present in the film composition in an amount of about 0.7% by weight of the film composition, (vi) sorbitol present in the film composition in an amount of about 26% by weight of the film composition, and (vii) acesulfam present in the film composition in an amount of about 1.4 percent by weight of the film composition.

The subject invention also provides a method for delivering laquinimod across the oral mucosa of a subject comprising administering to the oral mucosa of the subject an oral patch as described herein.

The subject invention also provides a method for treating a human subject afflicted with a form of multiple sclerosis, comprising periodically administering to the human subject an oral patch as described herein.

The subject invention also provides an oral patch as described herein for use in treating a human subject afflicted with a form of multiple sclerosis.

For the foregoing embodiments, each embodiment disclosed herein is contemplated as being applicable to each of the other disclosed embodiment.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Patent Application Publication No. 2005/0192315 and PCT International Application Publication No. WO 2005/074899, which are hereby incorporated by reference into this application.

Laquinimod can be administered alone but is generally mixed with a pharmaceutically acceptable carrier. Laquinimod can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. A dosage unit may comprise a single compound or mixtures of compounds thereof.

General techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage ̂Fozmu (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). These references in their entireties are hereby incorporated by reference into this application.

### TERMS

As used in this application, except as otherwise expressly provided herein, each of the following terms shall have the meaning set forth below.

As used herein, “laquinimod” means laquinimod acid or a pharmaceutically acceptable salt thereof.

As used herein, an “amount” or “dose” of an agent, e.g., laquinimod as measured in milligrams refers to the milligrams of the agent, e.g., laquinimod acid present in a preparation, regardless of the form of the preparation. A “dose of 0.6 mg laquinimod” means the amount of laquinimod acid in a preparation is 0.6 mg, regardless of the form of the preparation. Thus, when in the form of, e.g., a laquinimod sodium salt, the weight of the salt form necessary to provide a dose of 0.6 mg laquinimod would be greater than 0.6 mg (i.e., 0.64 mg) due to the presence of the additional salt ion.

Administration of different amounts of laquinimod using oral patches of the present invention can be accomplished by applying one, two, three, four or five oral patches at the same time or consecutively or by applying a portion of an oral patch. For example ½ of an oral patch can be obtained by cutting an oral patch once and ¼ of an oral patch can be obtained by cutting an oral patch twice.

Administration of an amount from about 0.2 to about 8 mg of laquinimod can be achieved using the oral patches of the present invention. For Example, administration of 0.25 mg laquinimod can be accomplished by applying ¼ of an oral patch containing 1 mg laquinimod and administration of 0.5 mg laquinimod can be accomplished by applying ½ of an oral patch containing 1 mg laquinimod. Likewise, administration of 1, 2, 3, 4 or 5 mg laquinimod can be accomplished, for example, by applying 1, 2, 3, 4 or 5 oral patches containing 1 mg laquinimod, respectively. Similarly, administration of 2 mg laquinimod can be accomplished, for example, by applying a single oral patch containing 2 mg laquinimod, or by applying 2 oral patches containing 1 mg laquinimod, etc.

As used herein, “saturation amount” of a substance in a composition means the amount above which the substance would no longer dissolve in the composition, and additional amounts of the substance will appear as a separate phase. Accordingly, where the composition as described herein contains a higher-than-saturation amount of laquinimod, the amount of laquinimod over the saturation amount will be present in the composition as non-dissolved laquinimod.

As used herein, a “unit dose”, “unit doses” and “unit dosage form(s)” mean a single drug administration entity/entities.

As used herein, “about” in the context of a numerical value or range means±10% of the numerical value or range recited or claimed. By way of example, about 1 mg includes the range of 0.90 mg-0.11 mg, i.e., 0.90, 0.91, 0.92, 0.93, 0.94, 0.95 . . . up to 0.11 mg. Accordingly, about 1 mg includes, in an embodiment, 1.00 mg.

As used herein, the term “composition”, as in pharmaceutical composition, is intended to encompass a product comprising the active ingredient(s) and the inert ingredient(s) that make up the carrier, as well as any product which results, directly or indirectly from combination, complexation, or aggregation of any two or more of the ingredients, or from dissociation of one or more of the ingredients, or from other types of reactions or interactions of one or more of the ingredients.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

As used herein, “film forming agents” are agents which form a matrix which allows for controlled release of an active ingredient. Film forming agents include, but are not limited to, Carbomer (sodium salt), polyethylene glycol, polyvinyl alcohol, microcrystalline cellulose, starch, hydroxypropyl methylcellulose, amylopectin, ethylcellulose, gelatine, hydroxypropylcellulose, hydroxyethylcellulose, methylcellulose, carboxymethylcellulose, gummi arabicum, xanthan gum, carrageen, ovidone and copovidon.

As used herein, “permeation enhancers” are agents which increase bioavailability of the active ingredient. Permeation enhancers include, but are not limited to, dimethyl sulfoxide (DMSO), n-Dodecyl nitrogen heterocyclic heptane-2-ketone, propylene glycol, isopropylmyristat, d,l-alpha-toccopherol and oleic acid.

Pharmaceutical compositions of the present invention can optionally comprise one or more colorants, flavors, and/or fragrances to enhance the visual appeal, taste, and/or scent of the composition. Suitable colorants, flavors, or fragrances are compatible with the ingredients of the pharmaceutical composition, i.e., they do not substantially reduce the solubility, the chemical stability, the physical stability or the biological activity of the pharmaceutical composition. In one embodiment, the pharmaceutical composition comprises a colorant, a flavor, and/or a fragrance. For example, the pharmaceutical composition comprises less than about 1 wt % (e.g., less than about 0.75 wt % or less than about 0.5 wt %) of each optional ingredient, i.e., colorant, flavor and/or fragrance, by weight of the composition. In another example, the pharmaceutical composition comprises less than about 1 wt % (e.g., less than about 0.75 wt % or less than about 0.5 wt %) of a colorant. In still another example, the pharmaceutical composition comprises less than about 1 wt % (e.g., less than about 0.75 wt % or less than about 0.5 wt %) of a blue colorant (e.g., FD&C Blue #1 and/or FD&C Blue #2 Aluminum Lake, commercially available from Colorcon, Inc. of West Point, Pa.)

As used herein, “flavourant” include sweeteners including but not limited to acesulfam, saccharin-sodium, aspartame, stevia, aspartam-acesulfam salt, cyclamat, neohesperidin, neotam, saccharin, sucralose, steviosid and thaumatin. Other suitable flavourants can include, for example, flavors, which are known to those of skill in the art, such as, for example, natural flavors, artificial flavors, and combinations thereof. Flavourants are compatible with the ingredients of the pharmaceutical composition, i.e., they do not substantially reduce the chemical stability, the physical stability, or the biological activity of the pharmaceutical composition. Flavoring agents may be chosen, e.g., from synthetic flavor oils and flavoring aromatics and/or oils, oleoresins, extracts derived from plants, leaves, flowers, fruits, and the like, and combinations thereof. Non-limiting examples of flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Suitable flavoring agents also include, for example, artificial, natural and synthetic flower derived or fruit flavors such as vanilla, ethyl vanillin, citrus oils (e.g., lemon, orange, tangerine, lime, and grapefruit), and fruit essences (e.g., natural and/or artificial flavor of apple, pear, peach, orange, grape, strawberry, raspberry, cherry, plum, pineapple, and apricot), and the like, and combinations thereof. The flavourants may be used in liquid or solid form and, as indicated above, may be used individually or in admixture. Other flavourants can include, for example, certain aldehydes and esters, e.g., cinnamyl acetate, cinnamaldehyde, citral diethylacetal, dihydrocarvyl acetate, eugenyl formate, p-methylamisol, and the like, and combinations thereof. They can be liquids or spray-dried, co-processed powders.

As used herein, colorants can include, but are not limited to, Annatto extract, Dehydrated beets (beet powder), Canthaxanthin, Caramel, β-Apo-8′-carotenal, β-Carotene, Cochineal extract, Carmine, Sodium copper chlorophyllin, Toasted partially defatted cooked cottonseed flour, Ferrous gluconate, Ferrous lactate, Grape color extract, Grape skin extract (enocianina), Synthetic iron oxide, Fruit juice, Vegetable juice, Carrot oil, Paprika, Paprika oleoresin, Mica-based pearlescent pigments, Riboflavin, Saffron, Titanium dioxide, Tomato lycopene extract; tomato lycopene concentrate, Turmeric, Turmeric oleoresin, FD&C Blue No. 1, FD&C Blue No. 2, FD&C Green No. 3, Orange B, Citrus Red No. 2, FD&C Red No. 3, FD&C Red No. 40, FD&C Yellow No. 5, FD&C Yellow No. 6, Alumina (dried aluminum hydroxide), Calcium carbonate, Canthaxanthin, Potassium sodium copper chlorophyllin (chlorophyllin-copper complex), Dihydroxyacetone, Bismuth oxychloride, Synthetic iron oxide, Ferric ammonium ferrocyanide, Ferric ferrocyanide, Chromium hydroxide green, Chromium oxide greens, Guanine, Pyrophyllite, Mica, Talc, Aluminum powder, Bronze powder, Copper powder, Zinc oxide, D&C Blue No. 4, D&C Green No. 6, D&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Red No. 39, D&C Violet No. 2, D&C Yellow No. 7, Ext. D&C Yellow No. 7, D&C Yellow No. 8, D&C Yellow No. 10 and D&C Yellow No. 11.

As used herein, a “perfusion enhancer” is an agent which increases blood flow to the capillary beds. Perfusion enhancers can include, but are not limited to, capsaicin and apitoxin and DMSO.

As used herein, a “humectant” is any one of a group of hygroscopic substances used to keep things moist. Fillers and humectants can include, but are not limited to, sorbitol, mannitol, isomalt, xylitol, glycerol, saccharose, dextrose and all other sugars and sugar replacer, except acesulfam and other sweeteners. In addition, fillers are selected so as to not make the film dry or too wet. A film which is too dry is too brittle and a film which is too wet is too sticky for the intended purpose.

As used herein, “a subject afflicted with multiple sclerosis” or “a subject afflicted with relapsing multiple sclerosis” means a subject who has been clinically diagnosed to have multiple sclerosis or relapsing multiple sclerosis (RMS), which includes relapsing-remitting multiple sclerosis (RRMS) and Secondary Progressive multiple sclerosis (SPMS).

As used herein, a subject at “baseline” is as subject prior to administration of laquinimod.

A “patient at risk of developing MS” (i.e. clinically definite MS) as used herein is a patient presenting any of the known risk factors for MS. The known risk factors for MS include any one of a clinically isolated syndrome (CIS), a single attack suggestive of MS without a lesion, the presence of a lesion (in any of the CNS, PNS, or myelin sheath) without a clinical attack, environmental factors (geographical location, climate, diet, toxins, sunlight), genetics (variation of genes encoding HLA-DRB1, IL7R-alpha and IL2R-alpha), and immunological components (viral infection such as by Epstein-Barr virus, high avidity CD4⁺ T cells, CM⁺ T cells, anti-NF-L, anti-CSF 114(Glc)).

“Clinically isolated syndrome (CIS)” as used herein refers to 1) a single clinical attack (used interchangeably herein with “first clinical event” and “first demyelinating event”) suggestive of MS, which, for example, presents as an episode of optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning, and 2) at least one lesion suggestive of MS. In a specific example, CIS diagnosis would be based on a single clinical attack and at least 2 lesions suggestive of MS measuring 6 mm or more in diameter.

As used herein, “administering to the subject” or “administering to the (human) patient” means the giving of, dispensing of, or application of medicines, drugs, or remedies to a subject/patient to relieve, cure, or reduce the symptoms associated with a condition, e.g., a pathological condition. The administration can be periodic administration. As used herein, “periodic administration” means repeated/recurrent administration separated by a period of time. The period of time between administrations is preferably consistent from time to time. Periodic administration can include administration, e.g., once daily, twice daily, three times daily, four times daily, weekly, twice weekly, three times weekly, four times weekly and so on, etc.

“Treating” as used herein encompasses, e.g., inducing inhibition, regression, or stasis of a disease or disorder, e.g., Relapsing MS (RMS), or alleviating, lessening, suppressing, inhibiting, reducing the severity of, eliminating or substantially eliminating, or ameliorating a symptom of the disease or disorder. “Treating” as applied to patients presenting CIS can mean delaying the onset of clinically definite multiple sclerosis (CDMS), delaying the progression to CDMS, reducing the risk of conversion to CDMS, or reducing the frequency of relapse in a patient who experienced a first clinical episode consistent with multiple sclerosis and who has a high risk of developing CDMS.

“Inhibition” of disease progression or disease complication in a subject means preventing or reducing the disease progression and/or disease complication in the subject.

A “symptom” associated with MS or RMS includes any clinical or laboratory manifestation associated with MS or RMS and is not limited to what the subject can feel or observe.

As used herein, “effective” or “therapeutically effective” when referring to an amount of laquinimod refers to the quantity of laquinimod that is sufficient to yield a desired therapeutic response. Efficacy can be measured by an improvement of a symptom of multiple sclerosis. Such symptoms can include a MRI-monitored multiple sclerosis disease activity, relapse rate, accumulation of physical disability, frequency of relapses, time to confirmed disease progression, time to confirmed relapse, frequency of clinical exacerbation, brain atrophy, neuronal dysfunction, neuronal injury, neuronal degeneration, neuronal apoptosis, risk for confirmed progression, visual function, fatigue, impaired mobility, cognitive impairment, brain volume, abnormalities observed in whole Brain MTR histogram, general health status, functional status, quality of life, and/or symptom severity on work.

It is understood that where a parameter range is provided, all integers within that range, tenths and hundredths thereof, are also provided by the invention. For example, “0.25-7.5 mg” includes 0.25 mg, 0.26 mg, 0.27 mg, 0.29 mg etc. up to 7.50 mg.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

Oral formulations of laquinimod in the form of capsules and tablets are disclosed in, e.g., PCT International Application Publication No. WO 2005/074899.

However, capsule and tablet formulations of laquinimod requires use of powder mixture in the manufacturing process, which may become inhomogeneous, particularly where the amount of laquinimod in the blend is low. Therefore, on production scale, content uniformity may be difficult to achieve.

Oral patch formulation overcomes the content uniformity problem because laquinimod is dissolved in a solution during the manufacturing process. Preparation of a fast-disintegrating orally-dissolving film employs laquinimod dissolved in solution, and provides a homogenous film.

### Materials

Laquinimod Na—Laquinimod sodium
Klucel EF (hydroxypropylcellulose or “HPC”)
PEG 1500 (polyethylene glycol)

### Avicel PH105 (Microcrystalline Cellulose or “MCC”)

Acesulfam K (acesulfam)
Kollidon VA 64 (copovidone).
HEC250G (hydroxyethylcellulose or “HEC”)
Mowiol (poilyvinylalcohol or “PVA”)

### Carbopol 971 Na (Carbomer)

### Example 1

Oral patches are prepared in accordance with the film composition as set forth in Table 1.

TABLE 1

| API + Excipient | [mg/10 cm²] | [%/DF] |
|---|---|---|
| Laquinimod Na | 0.75 | 1.07 |
| Klucel EF | 5.00 | 7.14 |
| (Hydroxypropylcellulose) | | |
| PEG 1500 | 7.25 | 10.36 |
| Avicel PH105 (MCC) | 31.00 | 44.29 |
| Sorbitol | 25.00 | 35.71 |
| Acesulfam K | 1.00 | 1.43 |
| TOTAL | 70.00 | 100.00 |

Laquinimod, polyethylene glycol (PEG), Sorbitol and acesulfam were dissolved in water on a magnetic stirrer. MCC was added while continuous stirring the suspension. After 15 minutes the HPC was added while continuous stirring the suspension. The suspension was stirred for minimum of 4 hours. The coating suspension was neutralised with a 15% NaOH solution. The suspension was coated with a coating knife onto a liner and dried in a cabinet dryer for 15 minutes at 50° C. The coating knife was adjusted so that after drying the film, the area weight was 70 g/m². The solid content of the suspension was 40%.

### Example 2

Oral patches are prepared in accordance with the film composition as set forth in Table 2.

TABLE 2

| API + Excipient | [mg/10 cm²] | [%/DF] |
|---|---|---|
| Laquinimod Na | 0.75 | 1.07 |
| Kollidon VA 64 (Copovidone) | 30.00 | 42.86 |
| PEG 1500 | 4.25 | 6.07 |
| Sorbitol | 18.00 | 25.71 |
| Rice starch | 14.00 | 20.00 |
| Acesulfam K | 1.00 | 1.43 |
| HEC250G | 2.00 | 2.86 |
| TOTAL | 70.00 | 100.00 |

Laquinimod, PEG, sorbitol, copovidone and, acesulfam were dissolved in water on a magnetic stirrer. Rice starch was added while continuous stirring the suspension. After 15 minutes the HEC was added while continuous stirring the suspension. The suspension was stirred for at least 4 hours. The coating suspension was neutralised with a 15% NaOH solution. The suspension was coated with a coating knife onto a liner and dried in a cabinet dryer for 15 minutes at 50° C. The coating knife was adjusted so that after drying the film, the area weight was 70 g/m². The solid content of the suspension was 40%.

### Example 3

Oral patches are prepared in accordance with the film composition as set forth in Table 3.

TABLE 3

| API + Excipient | [mg/10 cm²] | [%/DF] |
|---|---|---|
| Laquinimod Na | 0.75 | 1.07 |
| Mowiol (Polyvinylalcohol) | 30.00 | 42.70 |
| PEG 1500 | 6.00 | 8.54 |
| Sorbitol | 18.00 | 25.62 |
| Rice starch | 14.00 | 19.93 |
| Acesulfam K | 1.00 | 1.42 |
| Carbopol 971 Na (Carbomer) | 0.50 | 0.71 |
| TOTAL | 70.25 | 100.00 |

The PVA was dissolved in heated water and cooled down to room temperature after dissolving. Laquinimod, PEG, sorbitol, and acesulfam were added and stirred on a magnetic stirrer until the parts were dissolved. Rice starch was added while continuous stirring the suspension. After 15 minutes the Carbomer was added while continuous stirring the suspension. The suspension was stirred for minimum of 4 hours. The coating suspension was neutralised with a 15% NaOH solution. The suspension was coated with a coating knife onto a liner and dried in a cabinet dryer for 15 minutes at 50° C. The coating knife was adjusted so that after drying, the film the area weight was 70.25 g/m². The solid content of the suspension was 35%.

### Example 4

### Dissolution Test

Dissolution test was performed on oral patches prepared according to Examples 1-3 as follows:

### Dissolution Equipment:

### Dissolution Tester: Distek 5100, GP000001

### Sampler: Distek Evolution 4300, GP 000622

### Degasser: Riggtek Dissoprep X8, Gp000767

Dissolution Apparatus Paddle (Apparatus 2) with sinker
Dissolution Medium: 50 mM NaH2PO4 pH 6.8, degassed

### Volume: 1000 ml

### Temperature: 37° C.±0.5° C.

Stirrer speed: 75 rpm
Sampling points: 5, 10, 15, 20, 30, 60 minutes

### Preparation of the Dissolution Samples:

Laminate samples for dissolution testing were prepared according to the following protocol: Cut off a 10 qcm piece from the laminate using a circular cutter, remove and waste the release liner, roll the remaining laminate up, weigh it and put it into the sinker.

### Preparation of the Dissolution Medium:

Dissolution medium was prepared according to the following protocol: Weigh in 68.95 g NaH2PO4×H₂O and 9.6 g NaOH pellets and dissolve in 10 L of de-mineralized water. If necessary adjust the pH to 6.8±0.05.

### Results:

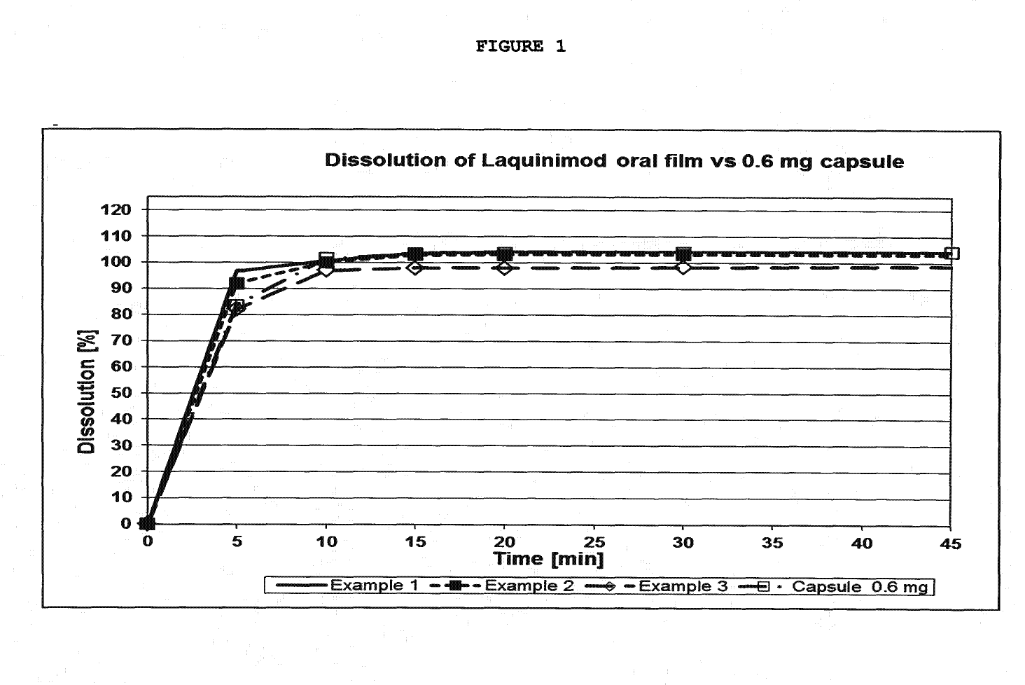

Dissolution results are shown in FIG. 1.

### REFERENCES

1. Bjartmar and Fox (2002) “Pathological mechanisms and disease progression of multiple sclerosis: therapeutic implication”, Drugs of Today. 38:7-29.
2. Brück (2011) “Insight into the mechanism of laquinimod action.” J Neurol Sci. 2011 Jul. 15; 306(1-2):173-9.
3. Brück et al. (2012) “Reduced astrocytic NF-kappaB activation by laquinimod protects from cuprizone-induced demyelination.” Acta Neuropathol. 124:411-424.
4. Brunmark et al. (2002) “The new orally active immunoregulator laquinimod (ABR-215062) effectively inhibits development and relapses of experimental autoimmune encephalomyelitis.” J Neuroimmunol. 130:163-172.
5. Comi et al. (2008) “Effect of laquinimod on MRI-monitored disease activity in patients with relapsing-remitting multiple sclerosis: a multicentre, randomised, double-blind, placebo-controlled phase IIb study”. Lancet. 371:2085-2092.
6. Comi et al. (2012) “Placebo-controlled trial of oral laquinimod for multiple sclerosis.” N Engl J Med. 366:1000-1009.
7. Dunitz. M. (1999) Multiple sclerosis therapeutics, Ed. Rudick and Goodkin. London: Taylor & Francis, 1999.
8. EMEA Guideline on Clinical Investigation of Medicinal Products for the Treatment of Multiple Sclerosis (CPMP/EWP/561/98 Rev. 1, November 2006).
9. Frohman et al. (2003) “The utility of MRI in suspected MS: report of the Therapeutics and Technology Assessment Subcommittee of the American Academy of Neurology”, Neurology. Sep. 9, 2003, 61(5):602-11.
10. Gasperini and Ruggieri (2009) “New oral drugs for multiple sclerosis.” Neurol Sci. 30 (Suppl 2):S179-183.
11. Gurevich et al. (2010) “Laquinimod suppress antigen presentation in relapsing-remitting multiple sclerosis: in-vitro high-throughput gene expression study.” J Neuroimmunol. 221:87-94.
12. Hohlfeld et al. (2000) “The neuroprotective effect of inflammation: implications for the therapy of multiple sclerosis”, J Neuroimmunol. 107:161-166.
13. Kurtzke J F. (1983) “Rating neurologic impairment in multiple sclerosis: an expanded disability status scale (EDSS)”, Neurology 33(11):1444-1452.
14. McDonald, (2001) “Guidelines from the International Panel on the Diagnosis of Multiple Sclerosis” Ann. Neurol. 50:121-127.
15. Multiple sclerosis: its diagnosis, symptoms, types and stages, 2003, albany.net/.about.tjc/multiple-sclerosis.html; What are the Types of Multiple Sclerosis?, 2005, <imaginis.com/multiple-sclerosis/types-of-ms.asp? mode=1>.
16. National Multiple Sclerosis Society Website “What We Know About Progressive-Relapsing MS (PRMS).” 28 January 13 Web. <http://www.nationalmssociety.org/about-multiple-sclerosis/progressive-ms/progressive-relapsing-ms/index.aspx>.
17. Neuhaus et al. (2003) “Immunomodulation in multiple sclerosis: from immunosuppression to neuroprotection”, Trends Pharmacol Sci. 24:131-138.
18. Noseworthy et al. (2000) “Multiple sclerosis”, N Engl J Med. 343:938-952.
19. PCT International Application Publication No. 2005/074899, published Aug. 18, 2005 (Jansson et al.).
20. PCT International Application Publication No. WO 2005/074899, published Aug. 18, 2005 (Jansson et al.).
21. Polman et al. (2005) “Diagnostic criteria for multiple sclerosis: 2005 revisions to the McDonald Criteria”, Annals of Neurology, 58(6):840-846.
22. Polman et al. (2005) “Treatment with laquinimod reduces development of active MRI lesions in relapsing MS.” Neurology. 64:987-991.
23. Polman et al. (2006) “A randomized, placebo-controlled trial of natalizumab for relapsing multiple sclerosis”, N Eng J Med. 354:899-910.
24. Poser et al. (1983) “New Diagnostic Criteria for Multiple Sclerosis: Guidelines for Research Protocols”, Annals of Neurology, March 1983, 13(3):227-230.
25. Product catalog published by Hanson Research (2001) (<http://www.prosense.net/files/MicroettePlus.pdf>, retrieved on Feb. 27, 2013).
26. Results of Phase III BRAVO Trial Reinforce Unique Profile of Laquinimod for Multiple Sclerosis Treatment; Teva Pharma, Active Biotech Post Positive Laquinimod Phase 3 ALLEGRO Results.
27. RTT News Article dated April 12, 11, entitled “Teva Pharma, Active Biotech Post Positive Laquinimod Phase 3 ALLEGRO Results”.
28. Runström et al. (2006) “Inhibition of the development of chronic experimental autoimmune encephalomyelitis by laquinimod (ABR-215062) in IFN-β k.o. and wild type mice” Journal of Neuroimmunology, 173(2006):69-78.
29. Sandberg-Wollheim et al. (2005) “48-week open safety study with high-dose oral laquinimod in patients”, Mult Scler. 11:S154 (Abstract).
30. Spain et al. (2009) “Recent developments in multiple sclerosis therapeutics.” BMC Medicine. 7:74.
31. The National MS Society (USA), The Disease Modifying Drug Brochure, Oct. 19, 2006.
32. Types of Multiple Sclerosis (MS), 2005, <themcfox.com/multiple-sclerosis/types-of-ms/types-of-multi-ple-sclerosis.htm>.
33. U.S. Patent Application Publication No. 2005-0192315, published Sep. 1, 2005 (Jansson et al.).
34. U.S. Patent Application Publication No. 2010-0322900, published Dec. 23, 2010 (Tarcic et al.).
35. U.S. Patent Application Publication No. 2011-0027219, published Feb. 3, 2011 (Tarcic et al.).
36. U.S. Patent Application Publication No. 2011-0034508, published Feb. 10, 2011 (Liat Hayardeny).
37. U.S. Patent Application Publication No. 2011-0217295, published Sep. 8, 2011 (Haviv and Tarcic).
38. U.S. Patent Application Publication No. 2011-0218179, published Sep. 8, 2011 (Haviv and Tarcic).
39. U.S. Patent Application Publication No. 2011-0218203, published Sep. 8, 2011 (Joel Kaye et al.).
40. U.S. Patent Application Publication No. 2012-0010238, published Jan. 12, 2012 (Fristedt).
41. U.S. Patent Application Publication No. 2012-0010239, published Jan. 12, 2012 (Piryatinsky et al.).
42. U.S. Patent Application Publication No. 2012-0142730, published Jun. 7, 2012 (Tarcic et al.).
43. U.S. Pat. No. 6,077,851, issued Jun. 20, 2000 (Bjork et al).
44. U.S. Pat. No. 7,589,208, issued Sep. 15, 2009 (Jansson et al).
45. U.S. Pat. No. 7,884,208, issued Feb. 8, 2011 (Frenkel et al.).
46. U.S. Pat. No. 7,989,473, issued Aug. 2, 2011 (Patashnik et al.).
47. U.S. Pat. No. 8,178,127, issued May 15, 2012 (Safadi et al.).
48. U.S. Pat. No. 8,252,993, issued Aug. 28, 2012 (Gant and Shahbaz).
49. Vollmer et al. (2011) “A placebo-controlled and active comparator phase III trial (BRAVO) for relapsing-remitting multiple sclerosis. 5th Joint Triennial Congress of the European and Americas Committees for Treatment and Research in Multiple Sclerosis.” Oct. 19-22, 2011; Amsterdam, The Netherlands: 148.
50, Wegner et al. (2010) “Laquinimod interferes with migratory capacity of T cells and reduces IL-17 levels, inflammatory demyelination and acute axonal damage in mice with experimental autoimmune encephalomyelitis.” J Neuroimmunol 227:133-43.
51. Yang et al. (2004) “Laquinimod (ABR-215062) suppresses the development of experimental autoimmune encephalomyelitis, modulates the Th1/Th2 balance and induces the Th3 cytokine TGF-beta in Lewis rats.” J Neuroimmunol. 156:3-9.

What is claimed:

1. An oral patch comprising:
a) a liner; and
b) a film composition thereon, the film composition comprising
i. laquinimod in an amount of about 0.1%-20% by weight of the film composition, and
ii. one or more film forming agents in a total amount of about 40%-90% by weight of the film composition.

2. The oral patch of claim 1, wherein laquinimod is present in the film composition in an amount of about 0.2%-10%, 0.6%-8% or 0.7%-1.5% by weight of the film composition.

3. The oral patch of claim 1 or 2, wherein the one or more film forming agents are present in the film composition in a total amount of about 60%-80% by weight of the film composition.

4. The oral patch of claim 3, wherein the one or more film forming agents are selected from the group consisting of Carbomer (sodium salt), polyethylene glycol, polyvinyl alcohol, microcrystalline cellulose, starch, hydroxypropyl methylcellulose, amylopectin, ethylcellulose, gelatine, hydroxypropylcellulose, hydroxyethylcellulose, methylcellulose, carboxymethylcellulose, gummi arabicum, xanthan gum, carrageen, povidone, copovidone.

5. The oral patch of claim 4, wherein the one or more film forming agents comprises:
a) carbomer (sodium salt), present in the film composition in an amount of about 0.1%-1% by weight of the film composition, or,
b) polyvinyl alcohol, present in the film composition in an amount of about 30%-50% by weight of the film composition, or
c) microcrystalline cellulose, present in the film composition in an amount of about 30%-50% by weight of the film composition, or
d) copovidone, present in the film composition in an amount of about 30%-50% by weight of the film composition, or
e) starch, present in the film composition in an amount of about 10%-30% by weight of the film composition, or
f) polyethylene glycol, present in the film composition in an amount of about 5%-15% by weight of the film composition, or
g) hydroxyethyl cellulose, present in the film composition in an amount of about 1%-10% by weight of the film composition, or
h) hydroxyethyl cellulose, present in the film composition in an amount of about 1%-10% by weight of the film composition.

6. The oral patch of any one of claims 1-5, wherein the film composition further comprises:
a) one or more fillers, present in the film composition in a total amount of about 10%-50% or about 20%-40% by weight of the film composition, and/or
b) one or more flavorants, present in the film composition in a total amount up to about 10% by weight of the film composition, and/or
c) one or more permeation enhancers, present in the film composition in a total amount up to about 10% by weight of the film composition, and/or
d) a pigment, present in the film composition in an amount up to about 5% by weight of the film composition, and/or
e) one or more humectants, present in the film composition in an amount up to about 5% by weight of the film composition.

7. The oral patch of claim 6, wherein the one or more fillers are selected from the group consisting of sorbitol, lactose, saccharose, sucrose, dextrose, isomalt calcium phosphate, calcium carbonate, calcium silicate, magnesium carbonate, magnesium oxide, glucopyranosyl mannitol and calcium sulfate.

8. The oral patch of claim 7, wherein the one or more fillers comprises sorbitol, present in the film composition in an amount of about 20%-40% by weight of the film composition.

9. The oral patch of any one of claims 1-8, wherein the flavorants are selected from the group consisting of acesulfam, saccharin-sodium, aspartame, stevia, spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, cassia oil, vanilla, ethyl vanillin, citrus oils, lemon oil, orange oil, tangerine oil, lime oil, grapefruit oil, apple flavor, pear flavor, peach flavor, orange flavor, grape flavor, strawberry flavor, raspberry flavor, cherry flavor, plum flavor, pineapple flavor, apricot flavor, cinnamyl acetate, cinnamaldehyde, citral diethylacetal, dihydrocarvyl acetate, eugenyl formate and p-methylamisol.

10. The oral patch of claim 9, wherein the one or more flavorants comprises acesulfam, present in the film composition in an amount of about 1%-3% by weight of the film composition.

11. The oral patch of any one of claims 1-10, wherein the one or more permeation enhancers are selected from the group consisting of DMSO, n-Dodecyl nitrogen heterocyclic heptane-2-ketone, propylene glycol, oleic acid, isopropylmyristat and d,l-alpha-toccopherol.

12. The oral patch of any one of claims 1-11, wherein the pigment is selected from the group consisting of titanium dioxide, talc and ferric oxide.

13. The oral patch of any one of claims 1-12, wherein the patch has an area about 5-15 cm² or about 10 cm².

14. The oral patch of any one of claims 1-13, wherein the patch comprises about 0.25-7.5 mg or about 0.75 mg laquinimod.

15. The oral patch of any one of claims 1-14, comprising about 0.5-5 mg laquinimod per 10 cm² of patch area, or comprising about 0.75 mg laquinimod per 10 cm² of patch area.

16. The oral patch of any one of claims 1-15, wherein the liner is a polyethylene terephthalate (PET) liner.

17. The oral patch of any one of claims 1-16, wherein the amount of laquinimod present in the pharmaceutical composition is a least laquinimod's saturation amount or wherein the amount of laquinimod present in the pharmaceutical composition is higher than laquinimod's saturation amount.

18. An oral patch comprising:
a) a PET liner, and
b) a film composition thereon, the film composition comprising
i. laquinimod in an amount of about 1% by weight of the film composition,
ii. hydroxypropylcellulose present in the film composition in an amount of about 7% by weight of the film composition,
iii. polyethylene glycol present in the film composition in an amount of about 10% by weight of the film composition,
iv. microcrystalline celluose present in the film composition in an amount of about 44% by weight of the film composition,
v. sorbitol present in the film composition in an amount of about 36% by weight of the film composition, and
vi. acesulfam present in the film composition in an amount of about 1.4% by weight of the film composition.

19. An oral patch comprising:
a) a PET liner; and
b) a film composition thereon, the film composition comprising
i. laquinimod in an amount of about 1% by weight of the film composition,
ii. copovidone present in the film composition in an amount of about 43% by weight of the film composition,
iii. polyethylene glycol present in the film composition in an amount of about 6% by weight of the film composition,
iv. starch present in the film composition in an amount of about 20% by weight of the film composition,
v. hydroxyethylcellulose present in the film composition in an amount of about 3% by weight of the film composition,
vi. sorbitol present in the film composition in an amount of about 26% by weight of the film composition, and
vii. acesulfam present in the film composition in an amount of about 1.4 percent by weight of the film composition.

20. An oral patch comprising:
a) a PET liner; and
b) a film composition thereon, the film composition comprising
i. laquinimod in an amount of about 1% by weight of the film composition,
ii. polyvinylalcohol present in the film composition in an amount of about 43% by weight of the film composition,
iii. polyethylene glycol present in the film composition in an amount of about 9% by weight of the film composition,
iv. starch present in the film composition in an amount of about 20% by weight of the film composition,
v. carbomer present in the film composition in an amount of about 0.7% by weight of the film composition,
vi. sorbitol present in the film composition in an amount of about 26% by weight of the film composition, and
vii. acesulfam present in the film composition in an amount of about 1.4 percent by weight of the film composition.

21. A method for delivering laquinimod across the oral mucosa of a subject comprising administering to the oral mucosa of the subject an oral patch of any one of claims 1-20.

22. A method for treating a human subject afflicted with a form of multiple sclerosis, comprising periodically administering to the human subject an oral patch of any one of claims 1-20.

23. An oral patch of any one of claims 1-20 for use in treating a human subject afflicted with a form of multiple sclerosis.
